(19) United States
(12) Patent Application Publication
(10) Pub. No.: US 2009/0270420 A1
(43) Pub. Date: Oct. 29, 2009

(54) METHODS FOR INHIBITING CARDIAC PAI-1

(76) Inventor: Burton E. Sobel, Colchester, VT (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/302,721

(22) PCT Filed: Jun. 1, 2007
(86) PCT No.: PCT/US07/12951

§ 371 Date: Jun. 23, 2009

Related U.S. Application Data
(60) Provisional application No. 60/809,804, filed on Jun. 1, 2006.

(51) Int. Cl. A61K 31/4965; A61K 31/4439; A61K 31/155

(52) U.S. Cl. 514/255.06; 514/342; 514/635

(57) ABSTRACT

The invention relates to methods of inhibiting cardiac PAI-1 by administering a PAI-1 antagonist.

### FIELD OF INVENTION

The invention relates to methods of inhibiting PAI-1 by administering a PAI-1 antagonist.

### BACKGROUND OF THE INVENTION

Cardiovascular disease is the leading cause of death in patients with type 2 diabetes. While the correlation between type 2 diabetes and cardiovascular disease stems in part from common risk factors such as obesity, they are also related to one another by the plasminogen activator inhibitor type-1 (PAI-1) pathway.

PAI-1 inhibits plasminogen activators (PA), such as t-PA and u-PA, which convert plasminogen to plasmin. Then, plasmin dissolves the clotting agent fibrin by a process called fibrinolysis. Thus, PAI-1 inhibits fibrinolysis. Sobel, “The Potential Influence of Insulin and Plasminogen Activator Inhibitor Type 1 on the Formation of Vulnerable Atherosclerotic Plaques Associated with Type 2 Diabetes,” Proceedings of the Association of American Physicians, 111(4): 313-18 (1999). When PAI-1 is over-expressed, these clotting agents can accumulate, resulting in accumulation of fibrin and consequently fibrosis. Furumoto et al., “Loss of insulin receptor substrate-1 signaling induces the cardiovascular and proteo(fibrino)lytic system derangements typical of insulin resistance,” Coronary Artery Disease 16(2): 117-23 (2005).

Because PAI-1 expression is increased by high levels of insulin, patients with hyperinsulinemea have a high risk for cardiovascular disease, including both myocardial infarction as well as diastolic dysfunction. See Furumoto et al.; and Sobel, “The Potential Influence of Insulin and Plasminogen Activator Inhibitor Type 1 on the Formation of Vulnerable Atherosclerotic Plaques Associated with Type 2 Diabetes,” Proceedings of the Association of American Physicians, 111(4): 313-18 (1999). Over-expression of PAI-1 in vessel walls can precipitate the formation of plaques vulnerable to rupture. See Sobel, et al., “Intramural Plasminogen Activator Inhibitor Type-1 and Coronary Atherosclerosis,” Arterioscler Thromb Vasc Biol., 1979-89 (2003). Increased PAI-1 expression in the heart has been associated with altered ventricular remodeling, heart failure, and both increased and decreased cardiac fibrosis depending on the extent of concomitant activation of macrophages.

Known pharmaceutical treatments for type 2 diabetes include insulin sensitizers such as biguanides (e.g., metformin) and thiazolidinediones (TZDs). Metformin also confers cardiovascular benefits by lowering PAI-1 levels in the blood and improving fibrinolysis. See, e.g., U.S. Pat. Nos. 6,693,094; 5,814,670; and 6,951,890. However, because metformin carries a perceived risk of lactic acidosis, metformin is contraindicated in patients with congestive heart failure.

Because insulin resistance is often a precursor to diabetes, and consequently cardiovascular disease, there is a need to prevent cardiovascular disease in insulin resistant patients. The invention provides this and other applications of cardiac PAI-1 inhibition.

### BRIEF DESCRIPTION OF FIGURES

FIG. 1: Representative echocardiographic images from a normal C57BL6 mouse. Left, a short axis view at the mid-papillary muscle level; right, an M mode image demonstrating wall thickening with systole.

FIG. 2: The correlation (least squares regression line) between the echocardiographic score obtained as described in the text and percent infarct determined by assay of left ventricular CK in 13 C57BL6 mice (closed circles) and 9 IRS2+/− ApoE−/− mice (open circles) studied 2 weeks after induction of infarction (r=0.81).

FIG. 3: PAI-1 in left ventricular myocardium from three strains of mice subjected to infarction. Results are means±SE's. Thirteen C57BL6, 9 IRS2+/− ApoE−/−, and 2 PAI-1 knockout mice were studied. *=significantly greater than PAI-1 in hearts of normal C57BL6 mice (0.45±0.04, n=21) and that in hearts from sham operated animals (0.42±0.02, n=9) and significantly greater as well than PAI-1 in hearts of normal IRS2+/− ApoE−/− mice (0.50±0.03, n=8). **=significantly greater than PAI-1 in grossly normal zones of the same hearts. There was no detectable PAI-1 in the hearts of the two PAI-1 knockout mice.

FIG. 4 A, B, and C: Concentrations of glucose in plasma as a function of time in minutes after injection-i.p. of 1.5 g/kg body weight in three strains of mice. Disappearance rates are markedly attenuated in mice in the insulin resistant strains (A and C). Results are means±SE.

FIG. 5: Content of PAI-1 in extracts of hearts from mice in the three strains studied. Open bars indicate results from 10 week old mice; shaded bars indicate those from 20 week old mice. Results are means±SE.

### DETAILED DESCRIPTION OF THE INVENTION

In one embodiment, the invention provides methods for inhibiting cardiac PAI-1 by administering a PAI-1 antagonist. Cardiac PAI-1 refers specifically to PAI-1 expressed by and/or accumulated in cardiac tissue. These methods recognize the discovery that PAI-1 is over-expressed by and/or accumulates in cardiac tissue. In particular, it is discovered that cardiac PAI-1 content increases in response to age, insulin resistance, and myocardial infarction. Without being bound by theory, it is believed that inhibition of cardiac PAI-1 decreases the risk for cardiovascular disease by reducing cardiac PAI-1 levels or preventing an increase in cardiac PAI-1 levels.

Inhibiting cardiac PAI-1 can be accomplished by any one or more of numerous strategies. Exemplary strategies include, but are not limited to, inhibiting cardiac PAI-1 expression, inhibiting cardiac PAI-1 accumulation, inhibiting the binding of cardiac PAI-1 to one or more of its targets, and inhibiting the ability of cardiac PAI-1 to affect one or more of its targets.

Inhibiting cardiac PAI-1 expression includes, but is not limited to, reducing, preventing, retarding, or otherwise impeding the expression and/or synthesis of cardiac PAI-1. Because healthy heart tissue usually expresses little or no PAI-1, one embodiment provides a method for preventing PAI-1 expression by cardiac tissue. In another embodiment, the method comprises inhibiting the subcellular expression of PAI-1. PAI-1 synthesis is known to be stimulated by free fatty acids, triglycerides, angiotensin II, IL-6, TGF-beta, and TNF-alpha among other moieties, as well as by insulin under conditions such as insulin resistance and consequent compensatory hyperinsulinemia. Thus, inhibiting PAI-1 synthesis includes, but is not limited to, lowering free fatty acid concentrations in blood; lowering triglyceride concentrations in the blood; lowering concentrations of angiotensin II; lowering the activity of angiotensin II; inhibiting cytokines such as IL-6, TGF-beta, and TNF-alpha, and ameliorating insulin resistance.

Similarly, inhibiting cardiac PAI-1 accumulation includes, but is not limited to, reducing, preventing, retarding, or otherwise impeding the accumulation of cardiac PAI-1. In one embodiment, inhibition of PAI-1 accumulation comprises inhibiting the secretion of PAI-1 from a cell. In another embodiment, the method comprises reducing and/or preventing the accumulation of PAI-1 in the heart and/or of PAI-1 expressed by cardiac tissue.

Inhibiting cardiac PAI-1 also includes interfering with effective PAI-1 binding to one or more of its targets. PAI-1 generally binds to and inhibits plasminogen activators (PA) such as t-PA and u-PA. A PAI-1 antagonist can reduce, prevent, or otherwise interfere with PAI-1's ability to bind one of its targets. For example, neutralizing antibodies or a soluble receptor can be used to block binding of PAI-1 to PA. The PAI-1 antagonist can also reduce, prevent, or otherwise interfere with PAI-1's ability to affect (e.g., inhibit) a PA.

The methods of inhibiting cardiac PAI-1 include a step of administering an antagonist of PAI-1 or of its expression. The PAI-1 antagonist can be any agent that inhibits PAI-1 or its expression. Exemplary PAI-1 antagonists include, but are not limited to, insulin sensitizing agents, e.g., metformin and TZDs; anti-hyperlipidemic agents, e.g., fibrate, niacin, probucol, bile acid sequestrant; inhibitors of lipoprotein lipase; HMG-CoA reductase inhibitors, e.g., statins; ACE inhibitors, e.g., captopril and ramapril; angiotensin II receptor blockers, e.g., candesartan; cytokine antagonists, e.g., antagonists of IL-6, TGF-beta, and TNF-alpha, such as agents used to block TNF for rheumatoid arthritis, e.g., antibodies and soluble TNF receptor; and other PAI-1 inhibitors such as tiplaxtinin. See also U.S. Pat. No. 6,916,813, incorporated herein by reference. In one embodiment, the PAI-1 antagonist is an agent that decreases insulin levels, such as metformin.

The PAI-1 antagonist can be administered in an amount sufficient to inhibit PAI-1. This PAI-1-inhibiting dose can, but need not be the same as the dose optimal for other indications. For example, a PAI-1 inhibiting dose of metformin can, but need not be the same as the dose of metformin administered to improve glycemic control in patients with type 2 diabetes. The PAI-1 antagonist can be administered in a dose of about 1 mg to about 2000 mg, about 1 mg to about 1000 mg, about 1 to about 500 mg, and increments therein. In one embodiment, the PAI-1 antagonist is administered in an amount that is less than the amount used for a different approved indication. Thus, in another embodiment, the PAI-1-inhibiting dose is less than about 2000 mg, less than about 1000 mg, less than about 500 mg, less than about 100 mg, or increments therein. One of ordinary skill in the art can prepare pharmaceutical formulations comprising a PAI-1 antagonist and a pharmaceutically acceptable carrier.

Throughout this application, the term “increment” is used to define a numerical value in varying degrees of precision, e.g., to the nearest 10, 1, 0.1, 0.01, etc. The increment can be rounded to any measurable degree of precision, and the increment need not be rounded to the same degree of precision on both sides of a range. For example, the range 1 to 100 or increments therein includes ranges such as 20 to 80, 5 to 50, and 0.4 to 98. When a range is open-ended, e.g., a range of less than 100, increments therein means increments between 100 and the measurable limit. For example, less than 100 or increments therein means 0 to 100 or increments therein unless the feature, e.g., temperature, is not limited by 0.

In one embodiment, the invention provides a method for inhibiting cardiac PAI-1 comprising a) selecting a patient, and b) administering a PAI-1-inhibiting dose of a PAI-1 antagonist to the patient. As demonstrated in the Examples below, it is discovered that cardiac PAI-1 content increases in response to age, insulin resistance, and myocardial infarction. Accordingly, in one embodiment, the patient is selected based on one or more of these factors.

In one embodiment, the patient is older than 30, 40, 50, 55, 60, or 75.

In one embodiment, the patient exhibits insulin resistance. Insulin resistance is accompanied by increased expression of PAI-1 in the vasculature and in diverse organs and tissues including liver and adipose tissue, but to the best of our knowledge, its impact on cardiac expression of PAI-1 has not been previously elucidated. Insulin resistance can be as assessed by any means known in the art, e.g., hyperinsulinemic euglycemic clamp, homeostasis model assessment of insulin resistance (HOMA-IR), quantitative insulin sensitivity check index (QUICKI), frequently sampled intravenous glucose tolerance test (FSIVGTT), modified insulin suppression test, fasting insulin test, glucose-to-insulin ratio (GIR), and oral glucose tolerance test (OGTT). See, e.g., Stern et al., “Identification of Individuals With Insulin Resistance Using Routine Clinical Measurements,” Diabetes, Vol. 54 (February 2005). In one embodiment, the patient exhibits a HOMA-IR greater than about 3, 3.6, 4, 4.65, or 5. In another embodiment, the patient requires less than about 10, 8, 7.5, 7, 6, 5, 4, or 3 mg/min or increments therein of glucose during the last 30 minutes of a hyperinsulinemic euglycemic clamp test.

In one embodiment, the patient exhibits insulin resistance, but is not diabetic. In another embodiment, the patient is diabetic.

In one embodiment, the patient exhibits polycystic ovarian syndrome (PCOS).

In one embodiment, the patient exhibits one, two, three, or more criteria of syndrome X, also known as metabolic syndrome. The criteria of syndrome X include the criteria defined by the National Cholesterol Education Program (NCEP) Adult Treatment Panel III (ATP III), the American Heart Association, the National Heart, Lung, and Blood Institute, the World Health Organization, the European Group for the Study of Insulin Resistance (EGIR), and any other recognized set of criteria for diagnosis of syndrome X, all of whose diagnosis criteria are incorporated herein by reference. Criteria of syndrome X include, but are not limited to, a waist circumference of greater than about 40 inches for a man or greater than about 35 inches for a woman, a fasting triglyceride level greater than or equal to about 150 mg/dL, an high-density lipoprotein cholesterol level of less than about 40 mg/dL for a man or less than about 50 mg/dL in women, systolic blood pressure greater than or equal to about 130 mm Hg, diastolic blood pressure greater than or equal to about 85 mm Hg, a fasting glucose level greater than or equal to about 100 mg/dL, impaired glucose tolerance demonstrated by a glucose level greater than about 140 mg/dL two hours after administration of 75 g glucose, body mass index of more than about 25 kg/m², urinary albumin excretion level greater than or equal to 20 mcg/min, and family history of congestive heart failure. In one embodiment, the patient exhibits syndrome X. In another embodiment, the patient exhibits one or more of obesity, dyslipidemia, endothelial dysfunction, atherosclerosis, hypertension, and prothrombotic activity.

In one embodiment, the patient has not experienced heart failure, e.g., a myocardial infarction or heart failure with preserved systolic function, also known as “diastolic dysfunction,” wherein excessive fibrosis stiffens the heart and impedes the ventricle's ability to accept incoming blood effectively. In this embodiment, the administration of a PAI-1 antagonist can be used to prevent heart failure. The invention also provides a method of inhibiting fibrosis comprising administering a PAI-1-inhibiting dose of a PAI-1 antagonist. In another embodiment, the patient has experienced a myocardial infarction. In another embodiment, the patient exhibits diastolic dysfunction.

The methods can further include administering at least one active pharmaceutical agent in addition to the PAI-1 antagonist. Exemplary additional agents include, but are not limited to anti-arrhythmic agents, anti-diabetic agents, anti-hyperglycemic agents, anti-obesity agents, lipid modulating agents, anti-hypertensive agents, anti-osteoporosis agents, and additional PAI-1 antagonists. In one embodiment, the additional agent is an angiotensin converting enzyme inhibitor, an angiotensin receptor blocker, or a statin. In another embodiment, the additional agent is a sulfonylurea compound; e.g., glipizide or glyburide; a thiazolidinedione; or another insulin sensitizer, e.g., rosiglitazone maleate.

The embodiments described herein are illustrative only, and other variations, substitutions, and modifications will be apparent to one of ordinary skill in the art. Although a feature may be described with respect to a particular embodiment, each feature described herein may be used with any embodiment of the invention. The invention is further described by the following non-limiting examples.

### EXAMPLES

### Example 1

### Insulin Resistance Increases PAI-1 in the Heart

This study was designed to determine whether insulin resistance increases expression of plasminogen activator inhibitor type-1 (PAI-1) in the heart subjected to acute myocardial infarction (MI). Studies were performed in 22 mice with and 38 without myocardial infarction. Insulin resistance in transgenic animals genetically rendered insulin resistant was confirmed with the use of intraperitoneal glucose tolerance tests. Myocardial infarction was induced by coronary ligation, verified echocardiographically, and quantified by assay of depletion of creatine kinase (CK) from the left ventricle 2 weeks later. PAI-1 increased markedly in zones of infarction to 10.4±2.1 (SF) and significantly more to 27.3±3.6 in normal and insulin resistant mice compared with 0.45±0.04 and 0.50±0.03 in normal myocardium. Thus, insulin resistance induced accumulation of PAI-1 in the heart, particularly in zones of infarction. Such increases may contribute to fibrosis and diastolic dysfunction typical late after infarction in patients with insulin resistance.

Transgenic mice were used in conformity with a protocol approved by the University of Vermont Institutional Animal Care and Use Committee and with adherence to the NIH Principles of Animal Care. Apolipoprotein E knockout (ApoE−/−) mice congenic with respect to the C57BL6 background were purchased from Taconic Corporation (Germantown, N.Y.).

To render mice insulin resistant insulin receptor substrate (IRS) 2 heterozygous, deficient mice (IRS2+/−) were rendered congenic on the C57BL6 background by backcrossing 10 times and were used for breeding. They were crossed with the ApoE−/− mice to generate insulin resistant IRS2+/− ApoE−/− mice. C57BL6 mice were used as controls. All mice were fed normal chow from weaning unless otherwise noted in which case a high fat diet was used (20% fat, 1.5% cholesterol; Teklad, Harlon Laboratories, Madison, Wis.).

Genotyping: The mice were genotyped by analysis of a 0.5 to 1 cm tail clip snipped at weaning. DNA extraction and PCR amplification were performed with the use of a Sigma RED Extract-N-Amp Tissue PCR Kit (product code: XNAT, Sigma Chemical Co., St. Louis, Mo.) consistent with instructions provided by the manufacturer. Primers were obtained from Invitrogen, Corp., Carlsbad, Calif. For IRS2−/−, the primers were: CTTGGCTACCATGTTATTGTC (5′); AGCTCTGGATTACTTTCCTAG (3′ wild type); and GCTACCCGTGATATTGCTGAAGAG (3′ encoding knockout/neomycin region). For ApoE−/−, the corresponding primers were: TGTCTTCCACTATTGGCTCG; CAGCTCTTTCACCCTCGGCA; and GTATCCATCATGGCTGATGC.

Metabolic Studies: To verify the presence of insulin resistance, we performed intraperitoneal (i.p.) glucose tolerance tests (GTT). We used a one compartment model since our objective was simply to verify the presence of insulin resistance. Absorption of glucose following i.p. injection is far from instantaneous and, accordingly, assumptions used in multi-compartmental models applied to intravenous GTTs are violated. The GTTs were performed by administrating 1.5 g/kg body weight of glucose i.p. followed by acquisition of tail nick samples for assay of plasma glucose at 30 minute intervals. Disappearance of glucose from blood during the interval of 60 to 120 minutes after injection was found to constitute the best fit mono-exponential portion of the curve of the log transformed data with linearity reflected by r values exceeding 0.98 in every case. The time required for 50% disappearance of glucose from the peak value to the trough or to 80 mg/dl, whichever was lower, was calculated as follows:

[G]ₜ_{_{1/2}}=[G]ₚe^{-kt}^{_{1/2}}
½=e^{-kt}^{_{1/2}}
k^{t}_{1/2}=In2
k^{t}_{1/2}=0.693
k=0.693/t_{1/2}
   in which [G]=concentration of glucose, [G]ₚ=peak concentration of glucose, [G]ₜ_{_{1/2}}=50% of [G]ₚ, k=disappearance rate (min⁻¹), t_{1/2}=time at which 50% of decline of concentration of glucose from [G]ₚ has occurred.

Induction of Myocardial Infarction: Myocardial infarction was induced in mice at 10 weeks of age following anesthesia initiated with 4% and maintained with 2% isoflurane, intubation, thoracotomy, resection of the pericardium, and occlusion of the left anterior descending coronary artery with an 8.0 suture on a tapered needled. The hearts were harvested 2 weeks later because at that time zones of infarction are clearly demarcated and readily apparent by gross visualization.

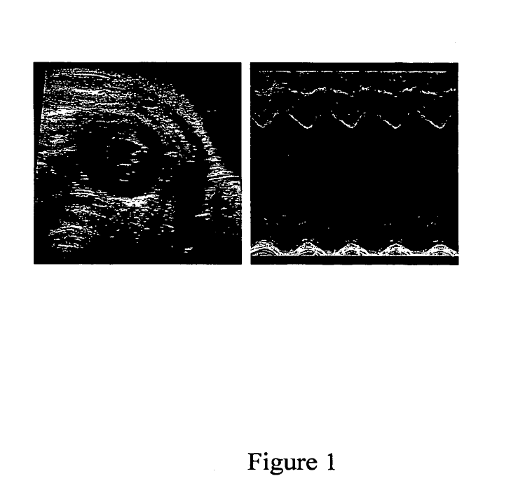

Ultrasonic Imaging of Mouse Hearts in vivo (Echocardiography): Anesthetized animals (4% followed by 2% isoflurane) were weighed and placed supine on to a 37° C. imaging platform. A VisualSonics (Toronto, Canada) Vivo 770 high-resolution ultrasound imaging system was used with a 45 MHz transducer. Imaging was performed through warmed ultrasound gel that had been centrifuged to remove air bubbles. Representative images are shown in FIG. 1. Image equality obtained with this system permitted reproducible assessment of segmented wall thickening and fractional shortening with virtually identical results consistently obtained by two blinded observers.

Echocardiographic images were obtained in parasternal long and short axis views. The parasternal long axis view was used to assess function of the apex. In the short axis view the heart was interrogated at 3 sites roughly dividing left ventricular chamber into thirds: basal (the junction between the chordae tendinae and the papillary muscle), mid (maximal papillary muscle mass), and apical.

Traditional echocardiographic M-mode measurements were performed at the short axis mid papillary muscle level for determination of anteroseptal and posterior wall thickness as well as internal dimensions during systole and diastole. Fractional shortening ([diastolic-systolic]/diastolic) was derived from the chamber dimensions.

A functional assessment of 13 segments patterned after the American Society of Echocardiography 17 segment model was performed as follows: each segment was scored based on wall thickening (1=normal, >25%; 2=hypokinetic, 10-25%; 3=akinetic, <10%. Normal function of all segments yielded a score of 13, each quarter of the short axis view providing 4 segments for analysis and the apex providing the 13^{th} segment. Echocardiography scores were determined independently by 2 observers, each masked with respect to estimates of infarct size and genotypes of the animals imaged.

Assessment of the Extent of Infarction: The extent of infarction was assessed with the use of a biochemical marker so that PAI-1 could be assayed biochemically in the same samples and to avoid potential pitfalls that can occur with or histological or histochemical determinations because of sampling bias, temporal-dependent changes in the evolution of histological and histochemical criteria, and factors influencing the activity of the dehydrogenases that interact with the dye among others. Assessment of the extent of infarction with the use of a biochemical marker of viable cells in homogenates of the whole left ventricle facilitates quantitative comparisons of hearts from different groups of animals. Accordingly, infarct size was assessed by assaying residual left ventricular (LV) CK, measured directly in homogenates. Loss of CK from myocardium correlates closely with extent of infarction measured with a variety of independent methods. The relationship underlies estimates of infarct size based on measurement of CK released into blood but is much more direct. The latter, in turn, correlate with scintigraphic and autopsy assessments in patients. Residual left ventricular content of other macromolecular markers such as troponin or lactate dehydrogenase (LDH) may not correlate directly with infarct size because of antibody reactions with proteolytic fragments in the case of troponin and red cell contributions to total activity in the case of LDH.

Left ventricular myocardial CK content was determined 2 weeks after the animals had been subjected to acute myocardial infarction as follows. The mice were killed humanely under isoflurane anesthesia by exsanguination. The hearts were excised, the atria and right ventricle trimmed and removed, the left ventricle frozen in isotonic saline and stored at −80° C. For analysis tissue from the center of homogeneous grossly evident infarction manifesting wall thinning and obvious pallor was homogenized as was normal left ventricular myocardium not supplied by the ligated left anterior descending coronary artery. Whole left ventricular content of analytes was calculated by pooling results from both homogenates. Tissue was homogenized after thawing in 5 mL polypropyline tubes in 50 mM Tris, 0.606 g/100 ml, 5 mM dithiothreitol (DTT), 0.077 g/100 ml chilled to 4° C. with the pH adjusted to 7.6 with HCl. Three bursts of a Polytron (Ultra Turrax T25) probe were applied at ½ maximum speed with a 15 second interval between bursts. After centrifugation in a microcentrifuge at maximum speed and 0 to 4° C. for 20 minutes the supernatant fluid was aspirated and transferred to another tube for analysis.

CK assays were performed on the same day as homogenization to avoid the potential impact of oxidants present in the tissue on CK activity, as were protein and PAI-1 assays. The extent of infarction (percent) was calculated based on observed total LV CK (IU/mg protein) CK in normal hearts that was found to average 8.1±0.6 (SD) IU/mg. Maximum CK depletion in homogeneous zones of infarction was found to yield residual CK averaging 1.4±0.1 IU/mg. Thus, percent infarction was calculated as =[(8.1-measured LV CK(IU/mg protein)]/6.7×100.

Assay of PAI-1 Protein: Assays of PAI-1 in homogenates of normal left ventricle and zones of infarction as well as in plasma were performed at room temperature with the use of a murine PAI-1 ELISA assay kit with monoclonal antibodies and reagents from Innovative Research, Inc., Southfield, Mich. TMB, a reagent containing both hydrogen peroxide and tetramethylbenzidine in an aqueous buffer, was added to elicit development of color at 450 nm read with a microplate reader. Standard curves were performed with the use of serial dilution of standards. Reactions were terminated with the addition of 50 μl of 1 N sulfuric acid. The amount of color developed reflected the concentration of total (free, latent, and complexed) PAI-1 in the sample.

Assay of Plasminogen Activator (PA) Activity: To determine whether changes in PAI-1 protein were paralleled by changes in PA activity in homogenates of whole left ventricles, normal myocardium, and zones of infarction, we assayed PA activity with a chromogenic kinetic assay with substrate S2251 from Diapharma (West Chester, Ohio), and Glu-plasminogen from Enzyme Research Laboratories (South Bend, Ind.). Fibrin fragments were prepared from fibrinogen obtained from Enzyme Research Laboratories, and tissue-type plasminogen activator was obtained from Genentech, Inc. (South San Francisco, Calif.).

Statistics: Results were expressed as means±standard deviations unless noted otherwise. Comparisons of continuous data were made with the use of Student's t-tests with the Bonferroni correction for avoidance of a type 1 error. P values≦0.05 were considered to be significant.

Confirmation of Insulin Resistance: As shown in Table 1, the IRS2+/− ApoE−/− mice were verified to be insulin resistant. Thus, the calculated disappearance rate of glucose from blood was markedly and significantly less than that in C57BL6 mice.

TABLE 1
Estimated Disappearance Rates of Glucose in Blood

| Strain | n | k (min⁻¹) |
|---|---|---|
| C57BL6 | 4 | 0.029 ± 0.002 |
| IRS2+/− ApoE−/− | 4 | 0.007 ± 0.006 * |

Results are means ± SE's.
* P < 0.05 compared with the k value observed in C57BL6 mice

Myocardial and Plasma PAI-1 in Animals Without Infarction: As shown in Table 2, compared with controls undergoing no procedure, sham operated animals exhibited low and similar myocardial PAI-1 content.

TABLE 2
PAI-1 Content in the Heart

| | | | | Myocardial PAI-1 |
|---|---|---|---|---|
| Strain | Tissue | Procedure | n | (ng/mg protein) |
| C57BL6 | whole LV | non-operated | 21 | 0.45 ± 0.04 |
| C57BL6 | whole LV | sham operated | 9 | 0.42 ± 0.02 |
| IRS2+/− | whole LV | non-operated | 8 | 0.50 ± 0.03 |
| ApoE−/− | | | | |
| C57BL6 | grossly normal | coronary | 13 | 0.72 ± 0.05 * |
| | myocardium | ligation | | |
| IRS2+/− | grossly normal | coronary | 9 | 0.83 ± 0.04 * |
| ApoE−/− | myocardium | ligation | | |

Results are means ± SE's.
* = significantly increased compared with myocardial PAI-1 in sham operated or non-operated animals of the same genotype

The same was true of PAI-1 concentrations in plasma (Table 3).

TABLE 3
PAI-1 in Plasma

| | | | PAI-1 in Plasma | |
|---|---|---|---|---|
| Strain | Procedure | n | (ng/mg protein) | Diet |
| C57BL6 | non-operated | 12 | 1.70 ± 0.02 | normal chow |
| C57BL6 | sham operated | 5 | 1.60 ± 0.3 | normal chow |
| C57BL6 | coronary | 5 | 1.40 ± 0.3 | normal chow |
| | ligation | | | |
| IRS2+/− | coronary | 2 | 2.31 ± 0.85 | normal chow |
| ApoE−/− | ligation | | | |
| IRS2+/− | non-operated | 8 | 2.70 ± 1.3 | normal chow |
| ApoE−/− | | | | |

Results are means ± SD's.

Results in Animals Subjected to Myocardial Infarction (MI): Infarct size averaged 13±10 (SD) and 26±12 in C57BL6 (n=13) and IRS2+/− ApoE−/− (n=9) mice (Table 4). Only 1 of 10 IRS2+/−ApoE−/− and 2 of 15 C57BL6 failed to survive for 2 weeks. Rupture was seen in only 1 mouse in each of these two groups.

TABLE 4
Echocardiographic Data in Mice Subjected to Myocardial Infarction

| | LVEDD | LVESD | | DWT | SWT | DWT | | | | |
|---|---|---|---|---|---|---|---|---|---|---|
| Animal Genotype | (mm) | (mm) | FS | Ant (mm) | Ant (mm) | Post (mm) | SWT Post (mm) | Score | % Infarct (CK) | HR |
| IRS2+/−, ApoE−/− | 3.5 | 2.4 | 33% | 1.2 | 1.4 | 1.1 | 1.3 | 19 | 12 | 369 |
| IRS2+/−, ApoE−/− | 3.5 | 2.6 | 26% | 0.6 | 0.7 | 0.7 | 0.8 | 20 | 14 | 466 |
| IRS2+/−, ApoE−/− | 5.0 | 4.0 | 20% | 0.3 | 0.6 | 0.9 | 0.9 | 25 | 30 | 432 |
| IRS2+/−, ApoE−/− | 3.4 | 1.9 | 43% | 0.4 | 0.7 | 1.0 | 1.3 | 18 | 15 | 387 |
| IRS2+/−, ApoE−/− | 4.6 | 3.8 | 18% | 0.6 | 0.7 | 0.7 | 0.6 | 25 | 30 | 455 |
| IRS2+/−, ApoE−/− | 5.1 | 3.57 | 29% | 0.70 | 0.99 | 0.61 | 1.02 | 21 | 37 | 495 |
| IRS2+/−, ApoE−/− | 3.4 | 1.93 | 44% | 0.79 | 1.14 | 0.89 | 1.16 | 13 | 16 | 449 |
| IRS2+/−, ApoE−/− | 5.6 | 4.97 | 11% | 0.61 | 0.64 | 0.92 | 1.07 | 28 | 42 | 485 |
| IRS2+/−, ApoE−/− | 4.9 | 4.16 | 15% | 0.68 | 0.9 | 0.22 | 0.41 | 28 | 37 | 442 |
| mean | 4.3 | 3.3 | 26% | 0.7 | 0.9 | 0.8 | 0.9 | 22 | 26 | 442 |
| SD | 0.9 | 1.1 | 12% | 0.3 | 0.3 | 0.3 | 0.3 | 5 | 12 | 42 |
| C57/BI6 | 4.4 | 3.0 | 32% | 0.8 | 1.3 | 0.8 | 3.0 | 20 | 17 | 435 |
| C57/BI6 | 3.7 | 2.8 | 23% | 0.7 | 0.9 | 1.0 | 0.8 | 21 | 17 | 441 |
| C57/BI6 | 4.8 | 4.0 | 17% | 0.9 | 0.9 | 0.8 | 0.8 | 19 | 20 | 456 |
| C57/BI6 | 4.4 | 3.3 | 25% | 0.7 | 1.0 | 0.7 | 0.8 | 23 | 18 | 519 |
| C57/BI6 | 4.1 | 2.9 | 29% | 0.6 | 0.9 | 0.8 | 0.9 | 15 | 6 | 436 |
| C57/BI6 | 3.9 | 2.8 | 28% | 0.8 | 1.0 | 0.6 | 0.7 | 13 | 0 | 495 |
| C57/BI6 | 3.4 | 2.3 | 32% | 0.9 | 1.0 | 0.7 | 0.8 | 13 | 0 | 536 |
| C57/BI6 | 5.3 | 4.8 | 9% | 0.7 | 0.4 | 0.5 | 0.5 | 24 | 30 | 459 |
| C57/BI6 | 3.8 | 2.5 | 28% | 0.8 | 1.0 | 0.8 | 0.9 | 13 | 1 | 464 |
| C57/BI6 | 4.2 | 3.5 | 17% | 0.5 | 0.3 | 1.1 | 1.5 | 25 | 19 | 478 |
| C57/BI6 | 4.2 | 3.1 | 27% | 0.7 | 1.0 | 0.7 | 0.7 | 16 | 6 | 462 |
| C57/BI6 | 5.4 | 4.8 | 11% | 0.3 | 0.2 | 0.7 | 0.7 | 21 | 13 | 492 |
| C57/BI6 | 5.7 | 5.6 | 3% | 0.3 | 0.3 | 0.7 | 0.7 | 31 | 23 | 500 |
| mean | 4.4 | 3.5 | 22% | 0.7 | 0.8 | 0.8 | 1.0 | 20 | 13 | 475 |
| SD | 0.7 | 1.0 | 9% | 0.2 | 0.3 | 0.2 | 0.6 | 5 | 10 | 32 |

Abbreviations:
LVEDD and SD (left ventricular end diastolic and end systolic dimensions);
FS (fractional shortening);
DWT and SWT (diastolic and systolic wall thickness);
Ant and Post (anterior and posterior);
% Infarct (based on residual CK);
HR (heart rate)

CK content in the grossly normal, unblanched, thick walled left ventricular myocardium that was not supplied by the ligated coronary artery in C57BL6 and IRS2+/−ApoE−/− mice was similar averaging 8.1±0.6 IU/mg protein and 8.8±0.5 in IRS2+/−ApoE−/− mice. These values were not significantly different from those in grossly normal zones in hearts harboring MI in the two strains (8.1±0.8 and 7.7±0.6, respectively).

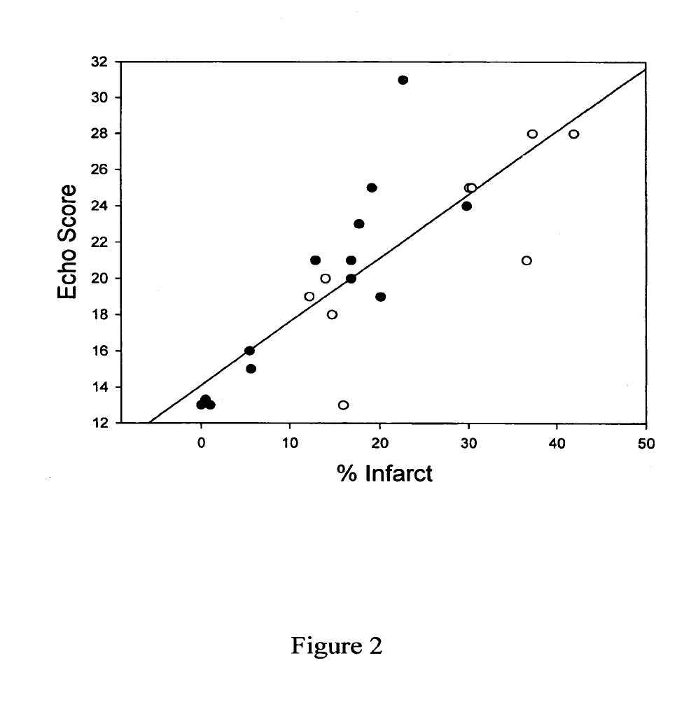

Echocardiographic score reflecting left ventricular dysfunction (Table 4) correlated with infarct size (r=0.81) calculated based on myocardial CK depletion 2 weeks after infarction in the 13 C57BL6 mice and 9 IRS2+/− ApoE−/− mice studied echocardiographically (FIG. 2). Fractional shortening correlated much less closely with infarct size than did score (r=0.50). This is not surprising because the extent to which the mid-ventricular region is affected by the induction of infarction will have a major influence on fractional shortening measured echocardiographically at the mid-papillary muscle region as will angulation of the echocardiographic beam at the time of interrogation.

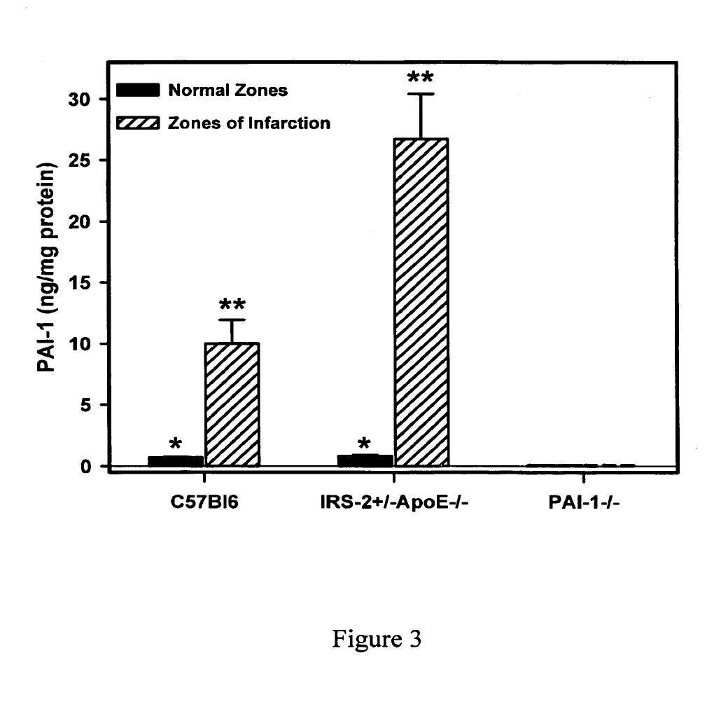

Myocardial Content of PAI-1 in Hearts from Mice with Myocardial Infarction: Two of 15 C57BL6 and 1 of 10 IRS2+/− ApoE−/− did not survive for the 2-week interval preceding planned echocardiography and tissue harvest. Their hearts were not included in the analyses. As can be seen in FIG. 3, in zones of infarction in C57BL6 control mice myocardial PAI-1 content was approximately 14 fold and significantly greater than that in grossly normal myocardium not supplied by the ligated coronary artery. Directionally similar results were seen in IRS2+/− ApoE−/− mice subjected to myocardial infarction (FIG. 3). Regions of myocardial infarction in the IRS2+/−ApoE−/− mice exhibited marked increases in PAI-1. Compared with results in C57BL6 control mice, the regions of infarction in the IRS2+/− ApoE−/− mice exhibited a 2.7-fold increase in PAI-1. Compared with PAI-1 content in grossly normal regions of the hearts from the same IRS2+/− ApoE−/− animals, the increase in PAI-1 was approximately 32 fold.

No PA activity was detected in homogenates of zones of infarction or in normal zones in mice of either strain consistent with the predominance of inhibition of PA activity attributable to PAI-1. The specific activity of plasminogen activator in tissues exceeds 200,000 IU/mg protein. The sensitivity of the assay used was such that as little as 10 IU of PA activity/mg protein would have been detectable.

Results obtained in this study indicate that PAI-1 increases markedly in infarct zones in both insulin sensitive and insulin resistant mice and that insulin resistance in IRS2+/−ApoE−/− transgenic mice leads to markedly greater increases in PAI-1 in zones of infarction in compared with the increases in controls that are not insulin resistant.

Previous studies show that PAI-1 co-localizes with fibrosis and that attenuation of the increased expression of PAI-1 by inhibition of angiotensin converting enzyme or angiotensin 2, known to mediate increased PAI-1 expression, diminishes coronary perivascular fibrosis in genetically obese mice. Increased PAI-1 expression in cardiac myocytes has been implicated as a profibrotic determinant after infarction. Increased PAI-1 expression occurs in activated macrophages. Most information available indicates that increased expression of PAI-1 predisposes to fibrosis by inhibiting degradation of extravascular fibrin that serves as a scaffold for developing fibrosis. However, some have found that deficiency of PAI-1 can cause cardiac fibrosis by reducing inhibition of urokinase-mediated macrophage infiltration that is profibrotic. Thus, effects of PAI-1 on fibrosis may depend on the company it keeps. When macrophage activation is prominent, increased PAI-1 may be antifibrotic. Under other conditions in which extravascular fibrin is prominent or another factor such as insulin resistance is present, it may be profibrotic.

Heart Failure and Insulin Resistance: Type 2 diabetes is known to be strongly associated with insulin resistance, implicated as an etiologic factor. It is also a powerful risk factor for and probable determinant of heart failure. An apparent diabetic cardiomyopathy acting synergistically with hypertension and coronary artery disease results in a high incidence of heart failure. Diastolic dysfunction, an early clinical manifestation of diabetic cardiomyopathy, is seen frequently. Increased ultrasonic backscatter indicative of changes consistent with deposition of collagen, accumulation of advanced glycation end products (AGEs), or interstitial edema is evident. Several studies have demonstrated fibrosis in hearts of insulin resistant animals. Of particular note, heart failure is emerging as a leading cause of death in patients with type 2 diabetes. We performed the present study to test the hypothesis that insulin resistance increases expression of PAI-1 in the heart, a potentially profibrotic phenomenon that could contribute to heart failure.

Increased PAI-1 in blood is seen with insulin resistance in human subjects. However, contributions of increased PAI-1 in blood were found in the infarcts to the PAI-1 found in the infarct and peri-infarct zones in the IRS2+/−ApoE−/− mice are likely to have been trivial in view of the actual concentrations seen in blood and the low regional myocardial blood volume in mice (11.8±0.8 volume percent).

These results show that insulin resistance markedly increases accumulation of PAI-1 in zones of infarction. Such changes may predispose to cardiac impairment typically seen late after infarction in patients with type 2 diabetes and insulin resistance.

### Example 2

### Increased PAI-1 in the Heart as a Function of Age

Heart failure is associated with advanced age and insulin resistance and is thought to be exacerbated by cardiac fibrosis. Plasminogen activator inhibitor type-1 (PAI-1) has been strongly implicated as a determinant of fibrosis in diverse organs and tissues. Its concentration is increased in blood, and its expression is increased in vessel walls in association with insulin resistance. This study was designed to determine whether expression of PAI-1 in the heart increases as a function of the age of 10 week old and 20 week old normal and insulin resistant transgenic mice thereby potentially predisposing to heart failure. Results obtained indicate that PAI-1 content increases significantly in the heart as a function of age by more than 60%. The increases are much greater than those that can be accounted for by the modest, and statistically insignificant increases in the concentrations of PAI-1 in plasma that were observed to occur as a function of age as well. Thus, PAI-1 increases in the heart is a function of age, occurs in insulin resistant and non-insulin resistant mice, and may contribute to fibrosis predisposing to heart failure associated with advanced age, particularly when insulin resistance is present.

Plasminogen activator inhibitor type-1 (PAI-1) has been implicated as a profibrotic factor diverse organs and tissues. Cardiac fibrosis appears to be one determinant of heart failure, particularly heart failure with preserved systolic function, known to be associated with advanced age, particularly in patients with insulin resistance, type 2 diabetes, and hypertension (Frohlich, 2001). The present study was designed to determine whether expression of PAI-1 in the heart increases with age in insulin resistant and non-insulin resistant mice thereby potentially predisposing to increased fibrosis, adverse ventricular remodeling after insults such as myocardial infarction, or both. It was performed also to determine whether studies designed to elucidate the role of PAI-1 expression with respect to cardiac fibrosis and its potential role in the evolution of heart failure require rigid adherence to stratification with respect to age of the animals and hearts characterized.

Mice of selected strains were used in conformity with a protocol approved by the University of Vermont Institutional Animal Care and Use Committee. Apolipoprotein E knockout mice (ApoE−/−), known to be prone to a form of atherosclerosis, congenic with respect to the C57BL6 genetic background were purchased from Taconic Corp. (Germantown, N.Y.). C57BL6 mice were used as controls. Insulin resistant mice were studied as well including heterozygous mice deficient in insulin receptor substrate 1 and 2 (IRS2+/− or IRS1+/−) and rendered congenic in the C57BL6 background through a minimum of 10 back crossings (Clough et al., 2005; Furumoto et al., 2005). All mice were fed normal chow from weaning unless noted otherwise in which case a high fat diet was used (20% fat, 1.5% cholesterol; Teklad, Harlon Laboratories, Madison, Wis.).

Genotyping: Insulin resistant transgenic mice were genotyped (Withers et al., 1999) by analysis of a 0.5 to 1 cm tail clip snipped at weaning with the use of scissors and forceps that were rinsed in 70% ethanol before and between different samplings. DNA extraction and PCR amplification were performed with the use of a Sigma RED Extract-N-Amp Tissue PCR Kit (product code: XNAT, Sigma Chemical Co., St. Louis, Mo.). Primers were obtained from Invitrogen, Corp., Carlsbad, Calif.

Extraction of DNA: Extraction buffer (50 μl) was pipetted into an Eppendorf tube. Tissue preparation solution in the kit (12.5 μl) was added to the same tube and mixed well by pipetting up and down. A tail tip (cut end down) was placed into the solution and maintained at 4° C. until extraction of DNA, usually within 30 minutes. For this purpose, samples were incubated at room temperature for 30 minutes; at 95° C. for 3 minutes; spiked with 50 μl of neutralization solution from the kit promptly; and mixed by vortexing. The neutralized tissue was stored at 4° C. or used immediately for performance of the polymerase chain reaction (PCR).

PCR Amplification: The RED Extract-N-Amp kit contains JumpStart Taq antibody for specific hot start amplication. Accordingly, PCR reactions were assembled at room temperature. A concentration of primer of 100 pM was used. The following reagents were added to the PCR tube: 10 μl RED Extract-N-Amp PCR reaction mixture, 4 μl tissue extract DNA, 2.5 μl downstream primer, 2.5 μl upstream primer, and purified water to make a final volume of 20 μl. After gentle mixing, thermal cycling was performed. Annealing temperatures depended on the specific characteristics of the primer used. Examples were: thermo cycling for ApoE−/− mice: 94° C. for 1 minute, 94° C. for 30 seconds, 62° C. for 1 minute, 72° C. for 2 minutes repeated for 30 cycles, 72° C. for 10 minutes and 4° C. until samples were removed; and thermo cycling for IRS1 and IRS2 mice: 94° C. for 1 minute, 94° C. for 30 seconds, 65° C. for 30 seconds, 72° C. for 1.5 minutes repeated for 30 cycles, and 72° C. for 10 minutes. The entire sample of amplified DNA was loaded on agarose gels for analysis as described previously (Withers et al., 1999).

Metabolic Studies were performed in the strains that were rendered insulin resistant, according to the method described in Example 1.

Impaired GTT can be attributable to impaired pancreatic beta cell function and peripheral insulin resistance in skeletal muscle, adipose tissue, and liver. Intraperitoneal insulin tolerance tests (ITTs) were performed to verify that it was not attributable exclusively to impaired β cell function. For this purpose, recombinant human insulin (Lilly, Indianapolis, Ind.) U100 was diluted in saline and injected i.p. at a dose of 0.75 U/kg body weight. Data were analyzed as described for the GTTs with the interval from the time of injection through 30 minutes used for acquisition of the slope of the monoexponential portion of the log transformed data curve based on the observed linearity.

Assay of PAI-1 Protein: Assays of PAI-1 in left ventricular homogenates and in plasma were performed at room temperature with the use of a murine PAI-1 ELISA assay kit with monoclonal antibodies and reagents from Innovative Research, Inc., Southfield, Mich. Whole, defatted left ventricles were fast frozen in isotonic saline, thawed and blotted immediately prior to assay, homogenized at 0-4° C. in 2 ml/100 mg 50 mM Tris, 5 mM dithiothreitol, pH 7.6 with the use of three 15-second bursts at maximum speed of a Polytron (Ultra-Turrax T25) probe (IKA Works, Inc., Wilmington, N.C.) with 15-second intervals between bursts. After centrifugation for 20 minutes at 0-4° C. in a microcentrifuge at maximum speed, murine PAI-1 was reacted with capture antibody (a mouse monoclonal to antibody (Declerck et al., 1995) against mouse PAI-1 raised in PAI-1 deficient mice) that had been coated and dried on a microtiter plate. The antibody binds free, latent, and complexed PAI-1. After washing, an anti-PAI-1 primary antibody (polyclonal anti-mouse PAI-1 raised in rabbits) was added, the excess washed away, and bound antibody, proportional to the total PAI-1 present in the samples, reacted with a secondary HRP-conjugated anti-rabbit antibody. After additional washing a reagent containing both hydrogen peroxide and tetramethylbenzidine in an aqueous buffer (TMB), was added. Development of color at 450 nm was read with a microplate reader. Standard curves were obtained with serially diluted standards. Reactions were arrested with 50 μl of 1 N sulfuric acid. Color developed was proportional to the concentration of total PAI-1 in the sample.

Assay of Plasminogen Activator (PA) Activity was assessed in myocardial homogenates, according the process described in Example 1.

Statistics: Significance (p<0.05) was assessed with the use of Student's t-tests with Bonferroni corrections.

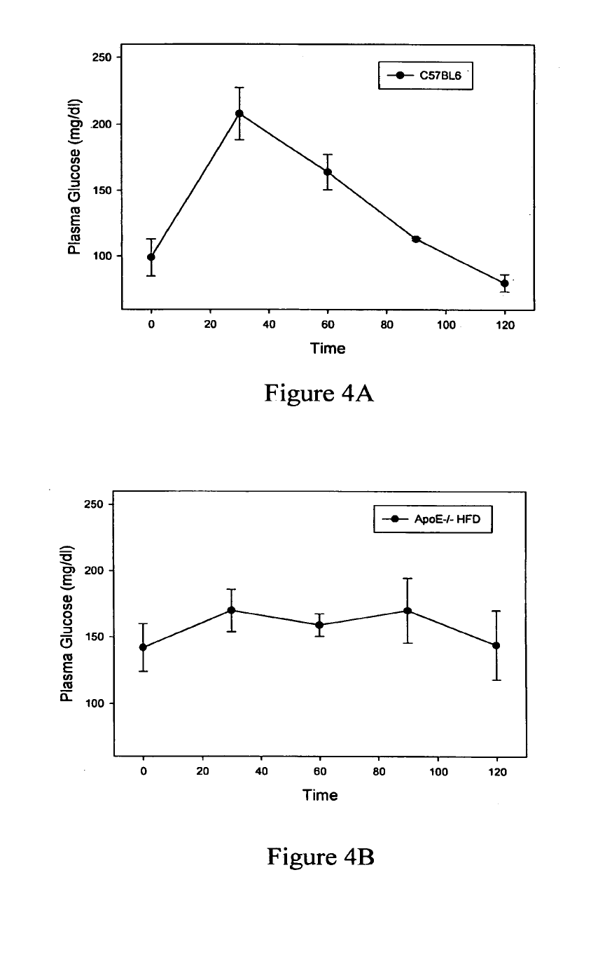

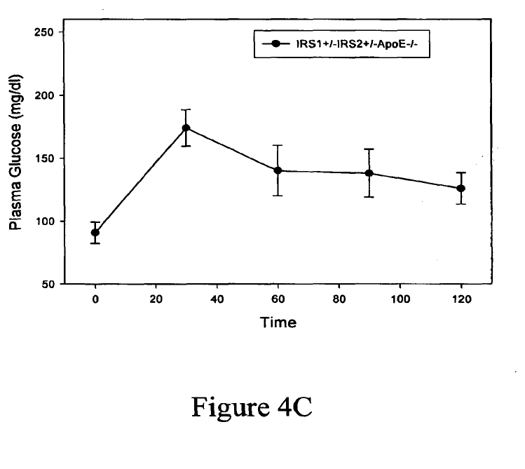

Confirmation of Insulin Resistance in Selected Strains: Insulin resistance was verified based on results of GTTs in ApoE−/− mice fed a high fat diet and in IRS1+/− IRS2+/− ApoE−/− mice (Table 5). The arithmetically plotted blood glucose data are shown in FIG. 4. The results of the ITTs confirmed the presence of insulin resistance. Thus, C57BL6 mice exhibited ITT k values that were significantly and 30% greater than those in the insulin resistant strains.

TABLE 5
Estimated Disappearance Rates of Glucose from Blood

| Strain | n | k (min⁻¹) |
|---|---|---|
| C57BL6 | 4 | 0.029 ± 0.002 |
| ApoE−/− (high fat diet) | 4 | 0.008 ± 0.005 * |
| IRS1+/− IRS2+/− ApoE−/− | 4 | 0.004 ± 0.001 * |

* P < 0.05 compared with the k value observed in C57BL6 mice. All mice were 20 weeks of age.
Results are means ± SDs.

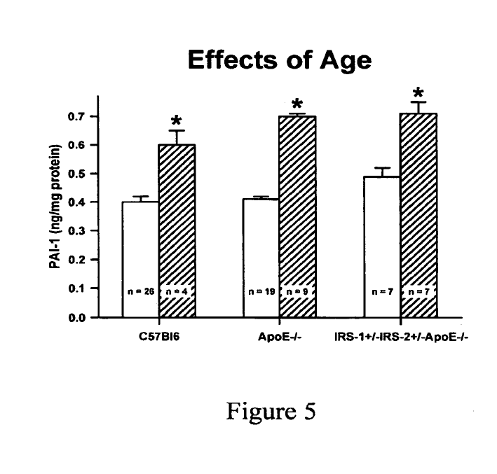

PAI-1 in the Heart: As shown in FIG. 5, C57BL6 mice of 10 weeks of age had significantly less PAI-1 in the heart compared with that in mice of the same strain of 20 weeks of age. Similar differences were evident in 10 week old ApoE−/− mice compared with 20 week old mice of the same genotype. Similarly, in the IRS1+/− IRS2+/− ApoE−/− mice of 10 weeks of age, PAI-1 content in the heart was significantly less than that in animals of the same genotype of 20 weeks of age. There was a trend, though differences were not significant, toward greater PAI-1 values in 20 week old insulin resistant compared with non-insulin resistant mice. Although the changes with age were modest in absolute terms, they were substantial in relative terms. Thus, the average percentage increase exceeded 60% (n=72 mice).

Concentrations of PAI-1 in plasma obtained from blood samples from the same strains of mice are shown in Table 6.

TABLE 6
PAI-1 in Plasma

| Strain | n | Age | PAI-1 (ng/mg protein) |
|---|---|---|---|
| C57BL6 | 17 | 10 weeks | 1.7 ± 0.3 |
| C57BL6 | 10 | 20 weeks | 2.4 ± 1.8 |
| ApoE−/− (normal chow) | 3 | 10 weeks | 4.1 ± 1.5 |
| ApoE−/− (high fat diet) | 13 | 10 weeks | 9.7 ± 9.6 * |
| ApoE−/− (high fat diet) | 9 | 20 weeks | 9.5 ± 3.3 * |
| IRS1+/− IRS2+/− ApoE−/− | 3 | 10 weeks | 1.8 ± 0.7 |
| IRS1+/− IRS2+/− ApoE−/− | 8 | 20 weeks | 3.5 ± 1.6 |

Significantly greater than values in C57BL6 mice of the same age
Results are means ± SDs.

Although a trend toward an increase in the concentration of PAI-1 in plasma as a function of age was evident in 20 week compared with 10 week old C57BL6 mice of the same genotype, it is unlikely that changes in concentrations of PAI-1 in blood accounted for the differences seen in analysis of homogenates of the hearts in view of the magnitude of the differences and the modest cardiac regional blood volume in mice (11.8±0.8 volume percent [Streif et al., 2005]). Thus, the maximal calculated contribution of increased plasma PAI-1 seen with increased age to measured PAI-1 content in the heart is less than 0.08 ng/mg protein. In addition, the comparability of concentrations of PAI-1 in plasma despite the age dependent difference in PAI-1 content in the heart in 10 and 20 week old ApoE−/− mice fed a high fat diet indicates that changes in PAI-1 in blood do not account for the changes in PAI-1 observed in the heart. The increased PAI-1 in plasma from ApoE−/− mice fed a high fat diet compared with those fed normal chow is consistent with the exacerbation of insulin resistance and consequent increased expression of PAI-1 (Furumoto, 2005) associated with high fat feeding in this strain (Cefalu et al., 2004). The lack of an increase in plasma PAI-1 in 10 week old IRS1+/− IRS2+/− ApoE−/− mice compared with that in 10 week old C57BL6 mice as opposed to the trend toward greater concentrations in the 20 week old animals is consistent with increasing effects of insulin resistance as a function of age typical of insulin resistant states.

No PA activity was detectable in homogenates of myocardium in mice from any of the strains studied. The sensitivity of the assay used was such that as little as 10⁻⁶ IU of PA activity/mg protein (i.e., <0.005 ng/mg protein) would have been detectable.

Results obtained in this study indicate that the content of PAI-1 in the heart increases with age in several strains of transgenic mice. As judged from phenomena in other organs, it appears likely that accumulation of PAI-1 may influence ventricular remodeling after coronary insults by predisposing to increased fibrosis. Thus, it may contribute to the development of heart failure with preserved systolic function typical of that seen with advanced age.

This study was designed to determine whether expression of PAI-1 in the heart increased with age. It was not designed to delineate the localization of PAI-1 in the normal heart or the heart subjected to an insult such as myocardial infarction. In future work, loci of sites accounting for age related increases in PAI-1 in the heart under specific conditions will require elucidation. The absolute magnitude of PAI-1 expression we observed was modest, but the relative increases with age were substantial. If these increases reflect changes in one or a small number of compartments such as vessel walls, inflammatory cells, or fibrous tissue consistent with our previous observations (Zaman et al., 2004) and those of others (Takeshita et al., 2004), the absolute magnitudes of increases in these specific sites may be large, and their biological impact may be considerable. In view of the present observations, studies designed for this purpose will require rigorous stratification with respect to age.

Plasminogen activator inhibitor type-1 (PAI-1) appears to predispose to fibrosis by inhibiting degradation of extravascular fibrin that serves as a scaffold for deposition of collagen (Sobel et al., 2003; Takeshita et al., 2004; Pinsky et al., 1998; Fogo, 2003; Kitching et al., 2003; Zaman et al., 2001; Zaman et al., 2004). Cardiac fibrosis has been implicated as a determinant of negative ventricular remodeling after myocardial infarction and congestive heart failure, particularly heart failure with preserved systolic function (Mizushige et al., 2000; Shimizu et al., 1993; Haider et al, 1978). Heart failure is a disease of the “very elderly,” and occurs frequently in association with a normal ejection fraction (Senni et al., 1998; van Veldhuisen et al., 1998; Chen et al., 2002; Kitzman, 2002; Miller and Missov, 2001). Even when systolic function is impaired, patients may be asymptomatic (Wang et al., 2003). The potential importance of ventricular fibrosis as a cause of heart failure in disorders such as hypertensive heart disease, has been recognized and emphasized (Frohlich, 2001; Owan and Redfield, 2005).

Previous studies delineate increased expression of PAI-1 in cells derived from subjects with premature aging (Werner syndrome) (Goldstein et al., 1994). Increases have been observed also as a function of age in the kidney subjected to vascular insults (Yamamoto et al., 1998). Increased concentrations of PAI-1 in blood are evident in sedentary women as a function of age (DeSouza et al., 1998) and in senescent human umbilical vein endothelial cells (West et al., 1996). In healthy male volunteers, euglobulin lysis time is prolonged apparently reflecting increased PAI-1 implicating impaired fibrinolysis as a function of advanced age (Gleerup and Winther, 1995). Analogous results as a function of age have been obtained in studies of the physiologic response to venous occlusion (Stegnar and Pentek, 1993).

PAI-1 in the heart has been found to be associated with mononuclear cells and cardiomyocytes juxtaposed to fibrous lesions induced by myocardial infarction (Takeshita et al., 2004) as well as in vessel wall constituents (Sobel et al., 2003). Gene expression of PAI-1 has been shown to increase with aging in multiple sites, tissues, and organs in a murine model of aging, the Klotho mutant (kl/kl) mice including plasma, kidney, cardiomyocytes, adrenal medullary cells, and smooth muscle and endothelial cells in Monckeberg's arteriosclerotic vessels (Takeshita et al., 2002). These observations were predicated on assay of PAI-1 mRNA. The present results, with which they are consistent, were predicated on assay of PAI-1 protein. We have found that increased PAI-1 in the heart accompanying conditions such as obesity and insulin resistance is associated with increased coronary perivascular fibrosis (Zaman et al., 2004). In the present study, PAI-1 in the heart increased with age in insulin resistant and non-insulin resistant mice. A trend toward greater PAI-1 content was seen with insulin resistance. Thus, the present results implicate increased expression of PAI-1 in the heart as a function of age in pathogenesis. The increased PAI-1 may constitute a profibrotic determinant potentially modifiable by pharmacologic interventions.

Results in the present study indicate that expression of PAI-1 in the heart increases with age. The age related increase in cardiac PAI-1 expression may predispose to the well recognized age dependent incidence of heart failure, particularly heart failure with preserved systolic function, thought to be attributable in part to cardiac fibrosis. Our results indicate also that studies addressing the potential profibrotic role of cardiac PAI-1 in the pathogenesis of heart failure must employ rigorous stratification with respect to the age of experimental animals or patients studied.

1. A method for inhibiting cardiac PAI-1 in a patient comprising administering a PAI-1-inhibiting dose of a PAI-1 antagonist or its expression to the patient.

2. (canceled)

3. (canceled)

4. (canceled)

5. A method for inhibiting cardiac PAI-1 comprising:
a. selecting a patient exhibiting insulin resistance, and
b. administering a PAI-1-inhibiting dose of a PAI-1 antagonist to the patient.

6. The method of claim 5, wherein inhibiting cardiac PAI-1 comprises reducing cardiac PAI-1 expression.

7. The method of claim 5, wherein inhibiting cardiac PAI-1 comprises preventing cardiac PAI-1 expression.

8. The method of claim 5, wherein inhibiting cardiac PAI-1 comprises reducing cardiac PAI-1 accumulation.

9. The method of claim 5, wherein inhibiting cardiac PAI-1 comprises preventing cardiac PAI-1 accumulation.

10. The method of claim 5, wherein inhibiting cardiac PAI-1 comprises inhibiting the binding of PAI-1 to one or more plasminogen activators.

11. The method of claim 5, wherein inhibiting cardiac PAI-1 comprises inhibiting the ability of PAI-1 to affect one or more plasminogen activators.

12. (canceled)

13. (canceled)

14. The method of claim 5, wherein the patient exhibits at least one of: obesity, dyslipidemia, endothelial dysfunction, atherosclerosis, hypertension, or prothrombotic activity.

15. The method of claim 5, wherein the patient exhibits at least one of: a waist circumference of greater than about 40 inches for a man or greater than about 35 inches for a woman, a fasting triglyceride level greater than or equal to about 150 mg/dL, an high-density lipoprotein cholesterol level of less than about 40 mg/dL for a man or less than about 50 mg/dL in women, systolic blood pressure greater than or equal to about 130 mm Hg, diastolic blood pressure greater than or equal to about 85 mm Hg, a fasting glucose level greater than or equal to about 100 mg/dL, impaired glucose tolerance demonstrated by a glucose level greater than about 140 mg/dL two hours after administration of 75 g glucose, body mass index of more than about 25 kg/m², urinary albumin excretion level greater than or equal to 20 mcg/min, or family history of congestive heart failure.

16. (canceled)

17. (canceled)

18. (canceled)

19. (canceled)

20. (canceled)

21. (canceled)

22. The method of claim 5, wherein the PAI-1 antagonist is an anti-hyperlipidemic agent, fibrate, niacin, an inhibitor of lipoprotein lipase, probucol, an ACE inhibitor, captopril, ramapril, an angiotensin 11 receptor blocker, candesartan, a cytokine antagonist, an IL-6 antagonist, a TGF-beta antagonist, a TNF-alpha antagonist, an insulin sensitizer, TZD, metformin, or tiplaxtinin.

23. The method of claim 22, wherein the PAI-1 antagonist is metformin.

24. The method of claim 23, wherein the dose of metformin is less than 500 mg.

25. The method of claim 5, further comprising administering at least one additional active pharmaceutical agent of: anti-arrhythmia agents, antidiabetic agents, anti-hyperglycemic agents, anti-obesity agents, lipid modulating agents, antihypertensive agents, anti-osteoporosis agents, or PAI-1 antagonists.

26. The method of claim 25, wherein the method comprises administering an angiotensin converting enzyme inhibitor, an angiotensin receptor blocker, or a statin.

27. The method of claim 25, wherein the method comprises administering a sulfonylurea compound or a thiazolidinedione.

28. The method of claim 27, wherein the sulfonylurea compound is glipizide or glyburide.

29. The method of claim 25, wherein the method comprises administering an insulin sensitizer.

30. The method of claim 29, wherein the insulin sensitizer is rosiglitazone maleate.

31. (canceled)

32. A method of inhibiting fibrosis comprising administering a PAI-1-inhibiting dose of a PAI-1 antagonist.

33. (canceled)

34. (canceled)


SEQUENCE LISTING (6 sequences)

SEQ ID NO: 1 - DNA, 21 bp, Artificial Sequence
  Chemically Synthesized
cttggctaccatgttattgtc

SEQ ID NO: 2 - DNA, 21 bp, Artificial Sequence
  Chemically Synthesized
agctctggattactttcctag

SEQ ID NO: 3 - DNA, 24 bp, Artificial Sequence
  Chemically Synthesized
gctacccgtgatattgctgaagag

SEQ ID NO: 4 - DNA, 20 bp, Artificial Sequence
  Chemically Synthesized
tgtcttccactattggctcg

SEQ ID NO: 5 - DNA, 20 bp, Artificial Sequence
  Chemically Synthesized
cagctctttcaccctcggca

SEQ ID NO: 6 - DNA, 20 bp, Artificial Sequence
  Chemically Synthesized
gtatccatcatggctgatgc